(19) United States
(12) Patent Application Publication
(10) Pub. No.: US 2022/0117581 A1
(43) Pub. Date: Apr. 21, 2022

(54) PATIENT MONITOR AND PHYSIOLOGICAL INFORMATION MEASUREMENT SYSTEM

(71) Applicant: NIHON KOHDEN CORPORATION, Shinjuku-ku, Tokyo (JP)

(72) Inventors: Mitsuhiro OURA, Tokorozawa-shi, Saitama (JP); Sou KUMAGAI, Tokorozawa-shi, Saitama (JP); Wataru MATSUZAWA, Tokorozawa-shi, Saitama (JP); Nobuyuki YASUMARU, Tokorozawa-shi, Saitama (JP); Kazuya NAGASE, Tokorozawa-shi, Saitama (JP); Hiroshi TORIGAI, Tokorozawa-shi, Saitama (JP); Naoki FUKUSHIMA, Tokorozawa-shi, Saitama (JP)

(21) Appl. No.: 17/424,652

(22) PCT Filed: Jan. 8, 2020
(86) PCT No.: PCT/JP2020/000308

§ 371 Date: Jul. 21, 2021

(30) Foreign Application Priority Data
Jan. 22, 2019 (JP) 2019-008780

(51) Int. Cl. A61B 8/00; A61B 8/14

(52) U.S. Cl. CPC A61B 8/463; A61B 8/464; A61B 8/54; A61B 8/14

(57) ABSTRACT

A patient monitor and physiological information measurement system are provided. The patient monitor for acquiring vital signals that are based on vital signs of a subject, and an ultrasonic image that is based on an ultrasonic wave which is transmitted to and received from the subject configured to be connectable to a plurality of displays, and includes a display controller that is configured to control the plurality of displays to display information including the vital signs in at least one of the displays.

### TECHNICAL FIELD

The presently disclosed subject matter relates to a patient monitor and physiological information measurement system that acquire vital signs and an ultrasonic image.

### BACKGROUND ART

A patient monitor for displaying vital signs of a subject is used in various medical sites such as an intensive-care unit (ICU), an operation room (OR), and an emergency medical care. Recently, a patient monitor to which an ultrasonic measurement apparatus for acquiring an ultrasonic image of the subject can be connected has been proposed. The patient monitor can display, together with vital signs, an ultrasonic image acquired by the ultrasonic measurement apparatus, and therefore allows various kinds of physiological information to be known at one glance.

In the case where the ultrasonic measurement apparatus is connected to the patient monitor, however, various kinds of physiological information relating to the vital signs and the ultrasonic image are mixedly displayed, and therefore there is a possibility that desired information cannot be adequately displayed.

As a technique for adequately displaying desired information, for example, Patent Literature 1 proposes a patient monitor that can provide a screen that is appropriate to the user. In the patient monitor, a plurality of modes in which vital signs and an ultrasonic image are displayed are set, and therefore desired information can be adequately displayed in accordance with the set mode.

### CITATION LIST

### Patent Literature

### PTL 1: Japanese Patent Publication No. 2017-86664

### SUMMARY OF INVENTION

### Technical Problem

The amount of information that is to be displayed on a patient monitor is increasing, and therefore a form in which a plurality of displays are disposed in a patient monitor is requested in an operation room and the like. In the case where a patient monitor that displays an ultrasonic image is used in this configuration (the form in which a plurality of displays are disposed), a configuration for rapidly knowing vital signs from various kinds of information that are displayed in the plurality of displays has room for improvement.

The presently disclosed subject matter is performed in order to solve the problem in the prior art. It is an object of the presently disclosed subject matter to provide a patient monitor and physiological information measurement system from which vital signs can be quickly known.

### Solution to Problem

A patient monitor of a first aspect of the presently disclosed subject matter is a patient monitor for acquiring vital signs that are based on vital signals of a subject, and an ultrasonic image that is based on an ultrasonic wave which is transmitted to and received from the subject. The patient monitor includes a display controller that is configured to be connectable to a plurality of displays, and that is configured to control the plurality of displays to display information including the vital signs in at least one of the displays.

A physiological information measurement system of a second aspect of the presently disclosed subject matter includes: an ultrasonic measurement apparatus that acquires receiving signals of an ultrasonic echo which is a base of an ultrasonic image, or an ultrasonic image; and the patient monitor.

According to the presently disclosed subject matter, the display controller is configured to be connectable to a plurality of displays, and is configured to control the plurality of displays to display information including vital signs in at least one of the displays. Therefore, it is possible to provide a patient monitor and physiological information measurement system from which vital signs can be quickly known.

### BRIEF DESCRIPTION OF DRAWINGS

FIG. 1 illustrates the configuration of a physiological information measurement system including a patient monitor of Embodiment 1 of the presently disclosed subject matter.

FIG. 2 is a block diagram illustrating the configuration of the physiological information measurement system.

FIG. 3 illustrates a manner of displaying an ultrasonic image in one display, and vital signs in another display.

FIG. 4 illustrates a manner of displaying vital signs in the one display, and the vital signs and the ultrasonic image in the other display.

FIG. 5 illustrates a manner of displaying vital signs and an ultrasonic image in the one display, and the vital signs in the other display.

FIG. 6 illustrates a manner of displaying an ultrasonic image in the one display, and vital signs and the ultrasonic image in the other display.

FIG. 7 illustrates a manner of displaying vital signs and an ultrasonic image in the one display, and the ultrasonic image in the other display.

FIG. 8 illustrates a manner of displaying vital signs and an ultrasonic image in both the one and other displays.

FIG. 9 is a block diagram illustrating the configuration of a physiological information measurement system of Embodiment 2.

FIG. 10(a) illustrates a manner of displaying main and auxiliary portions of an ultrasonic images separately and respectively in the one and other displays in Embodiment 4; and FIG. 10(b) illustrates a manner of displaying main and auxiliary portions of an ultrasonic images separately and respectively in the one and other displays in Embodiment 4.

FIG. 11 is a block diagram illustrating the configuration of a physiological information measurement system of modifications of Embodiments 1 to 4.

### DESCRIPTION OF EMBODIMENTS

Hereinafter, embodiments of the presently disclosed subject matter will be described with the accompanying drawings.

### Embodiment 1

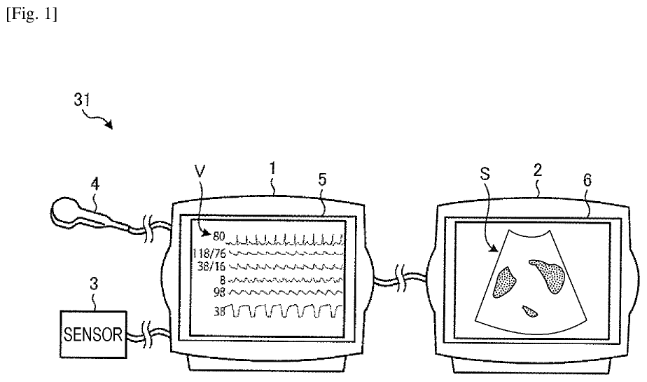

FIG. 1 illustrates the configuration of a physiological information measurement system 31 including a patient monitor of Embodiment 1 of the presently disclosed subject matter. The physiological information measurement system 31 may include the patient monitor 1, a display apparatus 2, a sensor 3, and an ultrasonic measurement apparatus 4.

The patient monitor 1 may include a display 5 in which vital signs V and an ultrasonic image S are to be displayed. In the patient monitor 1, connection ports such as connector jacks that are not illustrated are disposed, and the display apparatus 2, the sensor 3, and the ultrasonic measurement apparatus 4 are connected to the patient monitor through the connection ports. The patient monitor 1 measures the vital signs V based on vital signals acquired from the sensor 3 that is attached to the subject. The patient monitor 1 acquires the ultrasonic image S from the ultrasonic measurement apparatus 4 based on an ultrasonic wave that is transmitted to and received from the subject. The vital signs V and ultrasonic image S that are acquired in this way are displayed in the display 5.

The patient monitor 1 is configured by various medical devices that display the vital signs V, and may include, for example, a bedside monitor, a portable medical telemeter, a defibrillator having a function of measuring an electrocardiogram or the like, an electroencephalograph, an electromyograph, a respiratory apparatus, and an anesthesia apparatus. Examples of the vital signs V are the blood pressure, the body temperature, the respiration rate, the arterial oxygen saturation, an electrocardiogram, and the pulse rate. Examples of the ultrasonic image S are an A-mode image, a B-mode image, an M-mode image, and a D-mode image.

The display apparatus 2 is connected to the patient monitor 1 through a cable line, and may include a display 6 for displaying the vital signs V and ultrasonic image S that are supplied from the patient monitor 1.

The sensor 3 is configured by various sensors that acquire vital signals for producing the vital signs V, from the subject, and connected to the patient monitor 1 through a cable line. For example, the sensor 3 may include: a cuff used for measuring the blood pressure; electrodes (disposal electrodes, clip electrodes, and the like) used for measurement of an electrocardiogram, and the like; an SpO2 probe; a mask for measuring respiration; etc. The sensor 3 may include a sensor that acquires a vital signal by using an invasive method.

The ultrasonic measurement apparatus 4 transmits and receives an ultrasonic wave to and from the subject to acquire the ultrasonic image S of the interior of the living body of the subject, and is connected to the patient monitor 1 through a cable line. The ultrasonic measurement apparatus 4 is a device having a weight and size that allow the user such as the doctor to grip the device, and a form that is same as or similar to a probe head of a usual ultrasonic diagnosis apparatus.

At least one of the patient monitor 1 and the display apparatus 2 may be, for example, a ceiling suspended apparatus.

Next, the configurations of the patient monitor 1, the display apparatus 2, and the ultrasonic measurement apparatus 4 will be described in detail.

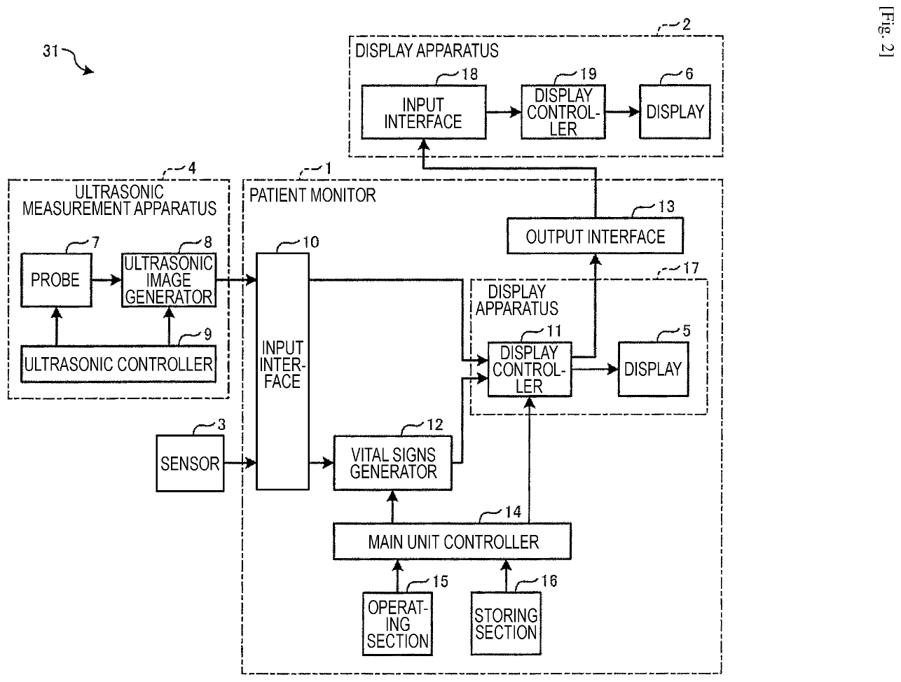

As illustrated in FIG. 2, the ultrasonic measurement apparatus 4 may include a probe 7, and an ultrasonic image generator 8 is connected to the probe 7. An ultrasonic controller 9 is connected to the probe 7 and the ultrasonic image generator 8.

The probe 7 transmits an ultrasonic wave to the subject, and receives an ultrasonic echo that is reflected from the body of the subject. The probe 7 outputs receiving signals of the ultrasonic echo to the ultrasonic image generator 8. For example, a probe of the convex type, the sector type, or the linear type may be used as the probe 7.

The ultrasonic image generator 8 matches and adds the receiving signals of the ultrasonic echo that are supplied from the probe 7, to form a sound ray signal, and produces the ultrasonic image S based on the sound ray signal.

The ultrasonic controller 9 controls various sections of the ultrasonic measurement apparatus 4 based on instructions input by the user from an operating section that is not illustrated.

The ultrasonic image generator 8 and the ultrasonic controller 9 are configured by a CPU and operation programs that cause the CPU to execute various operations. Alternatively, they may be configured by analog/digital circuits.

The patient monitor 1 may include an input interface 10. A display controller 11 is directly connected to the input interface 10, and also connected to the input interface 10 through a vital signs generator 12. The display controller 11 is connected to the display 5 and an output interface 13. A main unit controller 14 is connected to the vital signs generator 12 and the display controller 11. An operating section 15 and a storing section 16 are connected to the main unit controller 14.

The input interface 10 is configured by connection ports to which the sensor 3 and the ultrasonic measurement apparatus 4 are connected, and their peripheral circuits. The input interface 10 receives the vital signals that are measured by the sensor 3, and supplies the vital signals to the vital signs generator 12. The input interface 10 further receives the ultrasonic image S that is produced in the ultrasonic image generator 8, and supplies the image to the display controller 11.

The vital signs generator 12 produces the vital signs V based on the vital signals that are supplied from the input interface 10. The vital signs V can be produced in the forms of, for example, a waveform and a measurement value.

The display controller 11 is connected to the display 5, and further to the display apparatus 2 through the output interface 13, and controls the displays 5 and 6 so as to display the ultrasonic image S supplied from the input interface 10, and the vital signs V supplied from the vital signs generator 12. Here, a plurality of control rules corresponding to the display contents of the displays 5 and 6, the operations of the operating section 15, and the like are previously set in the display controller 11. The display controller 11 controls the displays 5 and 6 in accordance with the control rules. Specifically, the display controller 11 has a control rule for causing information including the vital signs V to be displayed in at least one of the displays 5 and 6. The display controller 11 produces a control signal corresponding to the control rule, and supplies, together with the vital signs V and the ultrasonic image S, to the display 5 and the output interface 13.

The display 5 may include a display device, and displays information such as the vital signs V and the ultrasonic image S in accordance with the control signal that is supplied from the display controller 11.

A display apparatus 17 is configured by the display controller 11 and the display 5.

The output interface 13 outputs the vital signs V, ultrasonic image S, and control signal that are supplied from the display controller 11, to the display apparatus 2.

The main unit controller 14 controls various sections of the patient monitor 1 based on instructions input from the operating section 15 by the user.

The operating section 15 is used for enabling the user to perform an input operation, and configured by buttons, knobs, a rotary selector, keys, or the like. Alternatively, the operating section 15 may be formed by a configuration that is integrated with the display 5, such as a touch panel.

The storing section 16 stores various programs that are to be used by the main unit controller 14, such as system software and application software. The storing section 16 may further store data such as measurement and set values of the blood pressure, the SpO2, and the like, and history and set values of the ultrasonic image. As the storing section 16, useful are recording media such as a hard disk drive, a USB memory, a flexible disk, an MO, an MT, a RAM, a CD-ROM, and a DVD-ROM.
The vital signs generator 12, the display controller 11, and the main unit controller 14 are configured by a CPU and operation programs that cause the CPU to execute various operations. Alternatively, they may be configured by digital circuits.

The display apparatus 2 may include an input interface 18. The display 6 is connected to the input interface 18 through a display controller 19.

The input interface 18 receives the vital signs V, the ultrasonic image S, and the control signals from the output interface 13 of the patient monitor 1, and supplies them of the display controller 19.

The display controller 19 controls the display 6 so as to display the vital signs V and the ultrasonic image S in accordance with the control signal supplied from the input interface 18. Namely, the display controller 11 controls the display 6 through the display controller 19.

The display 6 may include a display device, and displays information such as the vital signs V and the ultrasonic image S in accordance with the control signal that is supplied from the display controller 19.

Next, the operation of the embodiment will be described.

As illustrated in FIG. 2, first, the probe 7 of the ultrasonic measurement apparatus 4 transmits and receives an ultrasonic wave to and from the subject. Receiving signals of the ultrasonic echo that is received by the probe 7 are supplied from the probe 7 to the ultrasonic image generator 8. When receiving the receiving signals from the probe 7, the ultrasonic image generator 8 produces the ultrasonic image S based on the receiving signals. The produced ultrasonic image S is supplied from the ultrasonic image generator 8 to the display controller 11 through the input interface 10 of the patient monitor 1.

On the other hand, the vital signals of the subject are transmitted from the sensor 3, and supplied from the sensor 3 to the vital signs generator 12 through the input interface 10 of the patient monitor 1.

When the vital signals of the subject are input to the vital signs generator 12, the vital signs generator 12 sequentially produces the numerical values and waveforms of the vital signs V based on the vital signals. Then, the vital signs generator 12 supplies the produced numerical values and waveforms of the vital signs V to the display controller 11.

As described above, the ultrasonic image S that is produced in the ultrasonic image generator 8 is input to the display controller 11 through the input interface 10, and the vital signs V that are produced in the vital signs generator 12 are input to the display controller.

In accordance with the operation signal from the operating section 15 and the predetermined control rules, the display controller 11 then controls the display 5 and the display 6 of the display apparatus 2. In the display controller 11, here, a first control rule for displaying information including the vital signs V in at least one of the display 5 and the display 6 is previously set.

In the display controller 11, moreover, a second control rule for displaying information including the ultrasonic image S in the display that is other than the one display in which the information including the vital signs V is to be displayed may be previously set. In accordance with the first and second control rules, for example, the display controller 11 produces an internal control signal for displaying the vital signs V in the display 5, and an external control signal for displaying the ultrasonic image S in the display 6. The produced internal control signal and the vital signs V are supplied from the display controller 11 to the display 5, and the external control signal and the ultrasonic image S are supplied from the display controller 11 to the display 6 through the output interface 13, the input interface 18, and the display controller 19.

In this way, the internal control signal and the vital signs V are supplied to the display 5, and the external control signal and the ultrasonic image S are supplied to the display 6. As illustrated in FIG. 1, therefore, the vital signs V are displayed in the whole display 5, and the ultrasonic image S is displayed in the whole display 6.

As described above, the vital signs V and the ultrasonic image S are displayed in the different displays 5 and 6, respectively, and therefore both the vital signs V and the ultrasonic image S can be displayed in a large size. For example, the user can check in detail the ultrasonic image S.

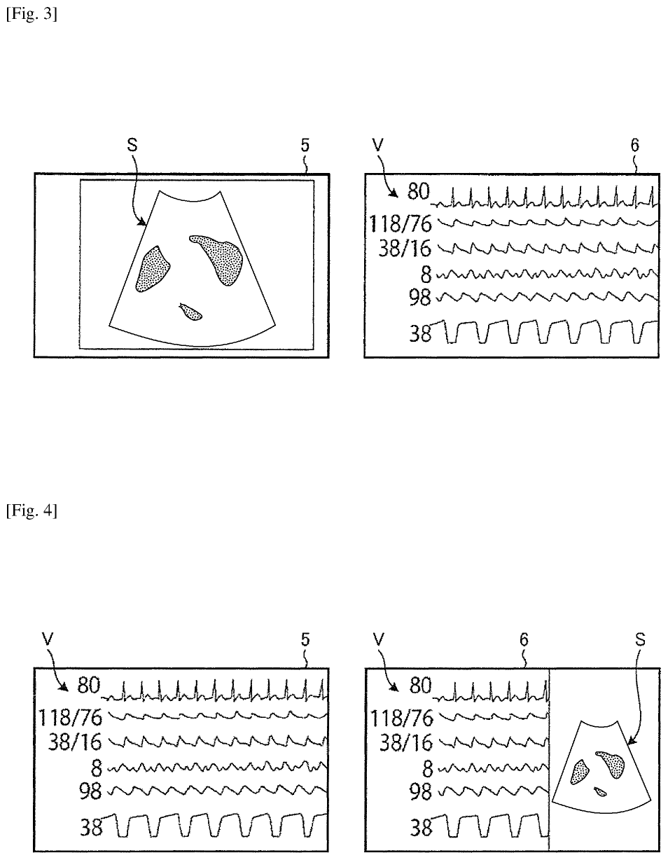

In the case where the user then operates the operating section 15 so that information including the ultrasonic image S is displayed in the display 5, the display controller 11 can then control so as to exchange information displayed in the display 5 with that displayed in the display 6, in accordance with the first and second control rules. Specifically, the display controller 11 produces an internal control signal for displaying the ultrasonic image S in the display 5, and an external control signal for displaying the vital signs V in the display 6. Then, the display controller 11 supplies the internal control signal and the ultrasonic image S to the display 5, and the external control signal and the vital signs V to the display 6 through the output interface 13, the input interface 18, and the display controller 19. As illustrated in FIG. 3, as a result, the ultrasonic image S is displayed in the whole display 5, and the vital signs V are displayed in the whole display 6.

In this way, the display controller 11 causes the information including the vital signs V to be displayed in at least one of the display 5 and the display 6, and therefore, in the case where the user wishes to urgently know the vital signs V, for example, the user can quickly know the vital signs V by simply moving the gaze from the display 5 to the display 6 without operating the operating section 15.

Moreover, the vital signs V and the ultrasonic image S are displayed in a large size in the different displays 5 and 6, respectively, and therefore it is possible to check in detail the vital signs V and the ultrasonic image S.

Alternatively, a third control rule for displaying information including the vital signs V in all of the displays 5 and 6 may be previously set in the display controller 11 in place of the first control rule. In accordance with the third control rule, for example, the display controller 11 can produce an internal control signal for displaying the vital signs V in the display 5, and an external control signal for displaying both the vital signs V and the ultrasonic image S in the display 6. As illustrated in FIG. 4, therefore, the vital signs V are displayed in the whole display 5, and the vital signs V and the ultrasonic image S are displayed in the display 6.

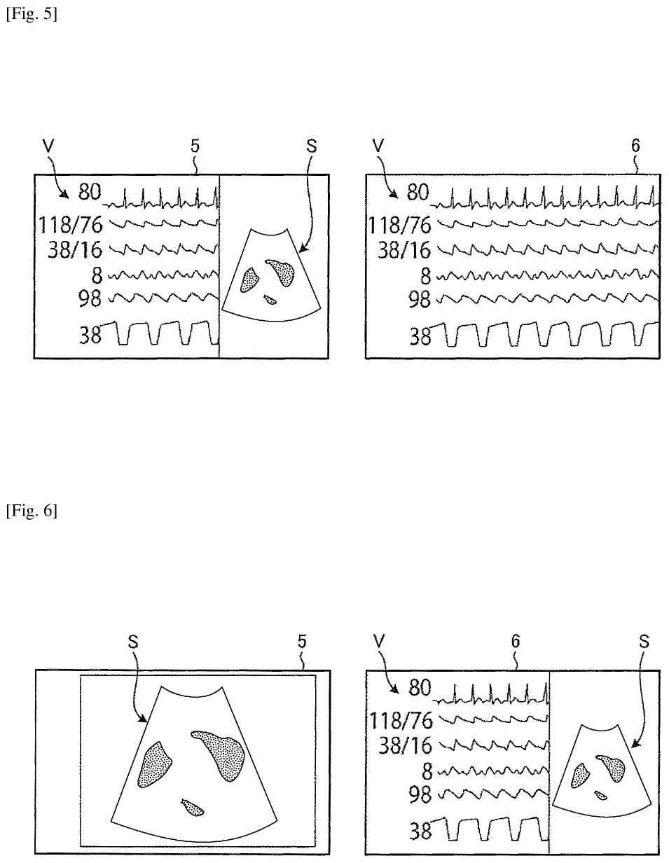

In the case where the user then operates the operating section 15 so that the ultrasonic image S is displayed in the display 5, the display controller 11 can cause the vital signs V and the ultrasonic image S to be displayed in the display 5, and the vital signs V to be displayed in the whole display 6 in accordance with the third control rule as illustrated in, for example, FIG. 5.

In this way, the display controller 11 causes the vital signs V to be displayed in all of the displays 5 and 6, and therefore, without searching the displays 5 and 6 in which the vital signs V are displayed, the user can quickly know the vital signs V by simply viewing one of the displays 5 and 6.

Alternatively, a fourth control rule for displaying information including the ultrasonic image S in all of the displays 5 and 6 may be previously set in the display controller 11. In accordance with the first and fourth control rules, for example, the display controller 11 can produce an internal control signal for displaying the ultrasonic image S in the display 5, and an external control signal for displaying both the vital signs V and the ultrasonic image S in the display 6. As illustrated in FIG. 6, therefore, the ultrasonic image S is displayed in the whole display 5, and the vital signs V and the ultrasonic image S are displayed in the display 6.

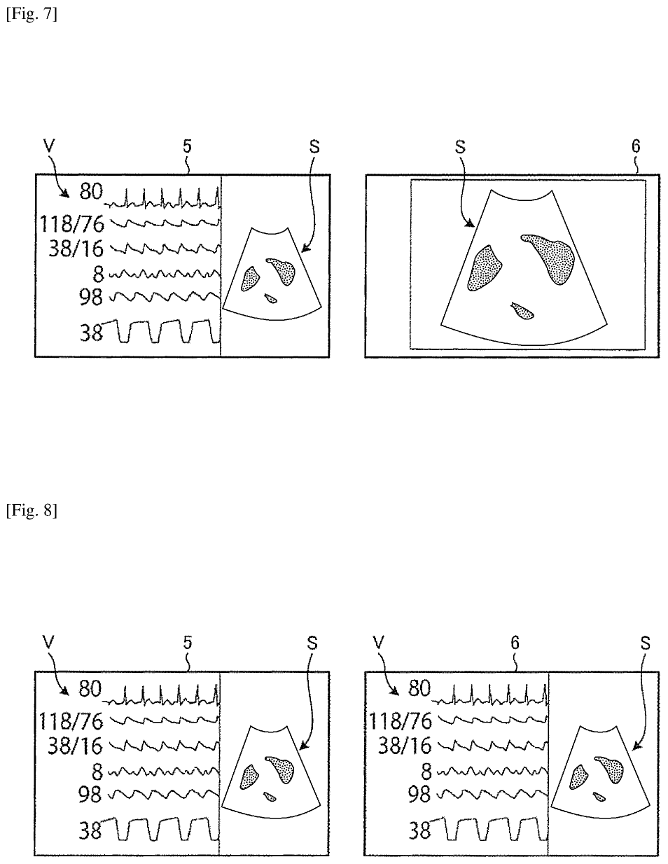

In the case where the user then operates the operating section 15 so that the ultrasonic image S is displayed in the whole display 6, the display controller 11 can cause the vital signs V and the ultrasonic image S to be displayed in the display 5, and the ultrasonic image S to be displayed in the whole display 6 in accordance with the first and fourth controls rules as illustrated in, for example, FIG. 7.

In this way, the display controller 11 causes the ultrasonic image S to be displayed in all of the displays 5 and 6, and therefore the user can quickly know the ultrasonic image S by simply viewing one of the displays 5 and 6.

Alternatively, a fifth control rule for displaying the vital signs V in all of the displays 5 and 6, and the ultrasonic image S in all of the displays 5 and 6 may be previously set in the display controller 11 in place of the first control rule. In accordance with the fifth control rule, for example, the display controller 11 can produce an internal control signal for displaying both the vital signs V and the ultrasonic image S in the display 5, and an external control signal for displaying both the vital signs V and the ultrasonic image S in the display 6. As illustrated in FIG. 8, therefore, the vital signs V and the ultrasonic image S are displayed in the displays 5 and 6.

In this way, the display controller 11 causes the vital signs V to be displayed in all of the displays 5 and 6, and the ultrasonic image S to be displayed in all of the displays 5 and 6, and therefore the user can simultaneously know the vital signs V and the ultrasonic image S by viewing any one of the displays 5 and 6.

According to the embodiment, the display controller 11 controls the display 5 and the display 6 so as to display information including the vital signs V in at least one of the displays 5 and 6, and therefore, without operating the operating section 15, it is possible to quickly know the vital signs V by simple viewing the displays 5 and 6.

In the display modes illustrated in FIGS. 3 to 8, it is a matter of course that various kinds of information (for example, the date, the patient name, and a set menu) other then the vital signs V and the ultrasonic image S may be adequately displayed.

According to the configuration in which the ultrasonic image S is displayed in one of the display sections such as in the configurations of FIGS. 3 to 8, it is possible to know not only the conditions of the vital signs V, but also the ultrasonic image S reflecting the condition of abdominal bleeding or the like. Therefore, it is possible to know more correctly the condition of the patient.

### Embodiment 2

In Embodiment 1 described above, in the case where a predetermined event occurs, preferably, the display controller 11 causes interest information corresponding to the predetermined event to be displayed in at least one of the displays 5 and 6.

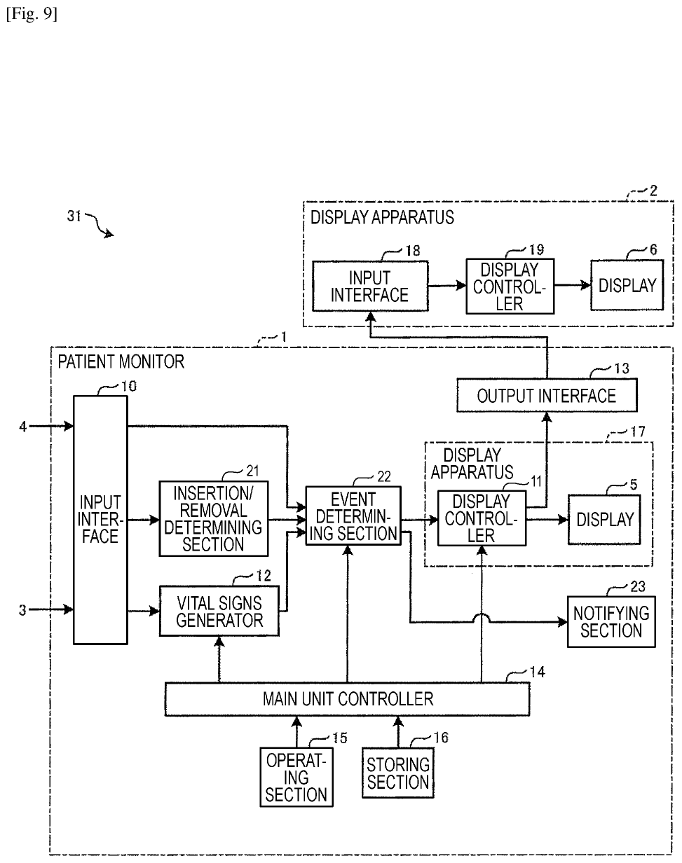

As illustrated in FIG. 9. for example, an insertion/removal determining section 21, an event determining section 22, and a notifying section 23 may be additionally disposed in Embodiment 1.

The insertion/removal determining section 21 is connected between the input interface 10 and the event determining section 22, and determines insertions and removals of the connectors of the ultrasonic measurement apparatus 4 and the sensor 3 to and from the input interface 10, based on inputs and outputs of the electrical signals to and from the input interface 10.

The event determining section 22 is connected between the input interface 10 and the display controller 11, and between the vital signs generator 12 and the display controller 11. The event determining section 22 is also connected to the main unit controller 14. The event determining section 22 determines whether the predetermined event occurs or not, based on the ultrasonic image S produced in the ultrasonic measurement apparatus 4, the vital signs V produced in the vital signs generator 12, the result of the determination in the insertion/removal determining section 21, and the operation signal of the operating section 15.

The notifying section 23 notifies the user that the event occurs, based on the result of the determination in the event determining section 22.

According to the configuration, the ultrasonic image S that is produced in the ultrasonic measurement apparatus 4 is input to the event determining section 22 through the input interface 10, and the vital signs V that are produced in the vital signs generator 12 are input to the event determining section 22. Based on the electrical signals that are input to and output from the input interface 10, the insertion/removal determining section 21 determines insertions and removals of the connectors of the ultrasonic measurement apparatus 4 and the sensor 3. The result of the determination is output from the insertion/removal determining section 21, and input to the event determining section 22. The operation signal of the operating section 15 is supplied to the event determining section 22 through the main unit controller 14.

When one of the vital signs V is input, the event determining section 22 determines whether the value of the vital sign V is degraded or not, i.e., whether the value of the vital sign V is increased or decreased by a predetermined value or more with respect to a reference value or not. The event determining section 22 further determines whether the value of the vital sign V rapidly changes or not. When the ultrasonic image S is input, the event determining section 22 further determines whether partial variation of the ultrasonic image S occurs, such as whether partial variation is caused in the ultrasonic image S by bleeding or the like or not. When the result of the determination in the insertion and removal determining section 21 is input, the event determining section 22 further determines whether the connector of the ultrasonic measurement apparatus 4 is connected to the input interface 10 or not. When the operation signal of the operating section 15 is input, the event determining section 22 further determines whether the operating section 15 is operated or not.

As described above, the event determining section 22 determines whether a predetermined event such as variation of the vital signs V, partial variation of the ultrasonic image S, an insertion of the ultrasonic measurement apparatus 4, or an operation of the operating section 15 by the user occurs or not, and supplies the result of the determination to the display controller 11 and the notifying section 23. The predetermined event is not limited to these events. Other examples of the predetermined event are an insertion/removal of the connector of the display apparatus 2 with respect to the output interface 13, an elapse of a predetermined time period (for example, a time period elapsing in the state where the signal from the ultrasonic measurement apparatus 4 is not received), and data reception from an external apparatus (for example, data of another patient monitoring system such as a medication administrating system).

When the determination result of the event determining section 22 is input to the display controller 11, the display controller 11 controls the display 5 and the display 6 of the display apparatus 2 based on the determination result. Here, a control rule for, in the case where the event determining section 22 determines that a predetermined event occurs, causing interest information corresponding to the predetermined event to be displayed in at least one of the displays 5 and 6 is previously set in the display controller 11. The interest information is information in which the user shows an interest in order to deal with the event. Examples of the interest information are the vital signs V, the ultrasonic image S, and an operation screen.

At this time, preferably, the display controller 11 changes information that is displayed in the displays 5 and 6, to contents that are desired by the user, in response to the event.

For example, a sixth control rule for, in the case where an event in which the vital signs V are rapidly degraded occurs, changing the display contents so that the measurement value of the blood pressure corresponding to the event is displayed in all of the displays 5 and 6 may be previously set in the display controller 11.

This allows the user to quickly know the measurement value of the blood pressure by simply viewing any one of the displays 5 and 6, and it is possible to adequately cope with the rapid degradation of the vital signs V. The cause of such variation of the display contents is not limited to degradation of the vital signs V. Even in the case where one of the vital signs V changes from an abnormal value to a normal value, for example, an identical or similar variation of the display contents may be performed. This causes the user to recognize that the vital sign V is directed to improvement by any cause (for example, a procedure).

Moreover, a seventh control rule for, in the case, when an event in which the vital signs V are rapidly degraded occurs, a further event in which the operating section 15 is operated by the user, such as that a button of the operating section 15 is pressed occurs, changing the display contents so that information including the vital signs V is displayed in all of the displays 5 and 6 as illustrated in, for example, FIG. 4 may be previously set in the display controller 11.

In the case where the vital signs V are rapidly degraded, therefore, the user is not required to perform as usual the operation of displaying the vital signs V, and the vital signs V are displayed by simply pressing any one of the buttons of the operating section 15. Therefore, the vital signs V can be quickly known.

The display controller 11 may change the display contents so that, in the case where an event in which the ultrasonic measurement apparatus 4 is connected to the input interface 10 occurs, the ultrasonic image S is displayed at least one of the displays 5 and 6 as illustrated in FIGS. 3 to 8, and, before the ultrasonic measurement apparatus 4 is connected to the input interface 10, and after the ultrasonic measurement apparatus 4 is disconnected from the input interface 10, information including the vital signs V is displayed in all of the displays 5 and 6.

As described above, the display contents of the displays 5 and 6 are changed in accordance with the behavior of the user, and therefore information desired by the user can be displayed more adequately in the displays 5 and 6.

Moreover, an eighth control rule for, in the case where an event in which the ultrasonic measurement apparatus 4 is connected to the input interface 10 occurs, changing the display contents so that an operation screen for operating the display of the ultrasonic image S is displayed in at least one of the displays 5 and 6 may be previously set in the display controller 11.

In this way, when the ultrasonic measurement apparatus 4 is simply connected to the input interface 10, the operation screen for operating the display of the ultrasonic image S is displayed, and therefore the user can rapidly start the operation of displaying the ultrasonic image S.

Furthermore, a ninth control rule for exchange information displayed in the display 5, and that displayed in the display 6 with each other so as to correspond to a predetermined event that is determined in the event determining section 22 may be previously set in the display controller 11.

For example, the display controller 11 can previously store characteristic information such as the sizes of the screens, installation places, and usage purposes of the displays 5 and 6. In the case where the vital signs V are displayed in the display 5 having a larger screen, and the ultrasonic image S is displayed in the display 6 having a smaller screen, when an event in which the ultrasonic image S is partly is changed occurs, the display controller 11 exchange the information sets respectively displayed in the displays 5 and 6 with each other so that the ultrasonic image S corresponding to the event is displayed in the display 5 having a larger screen in accordance with the ninth control rule.

As a result, the ultrasonic image S is displayed in the display 5 having a larger screen, and therefore the user can observe in detail the changed part of the ultrasonic image S.

Furthermore, a tenth control rule for highlighting interest information corresponding to the predetermined event that is determined in the event determining section 22, in at least one of the displays 5 and 6 may be previously set in the display controller 11.

In the case where an event indicating an abnormality of the subject such as degradation or sudden variation of the vital signs V occurs, for example, the event determining section 22 outputs the determination result to the notifying section 23 to notify the user that an abnormality occurs in the subject, based on the determination result. In the case where an event indicating an abnormality of the subject such as degradation or sudden variation of the vital signs V, such as where an event indicating an abnormality relating to arrythmia occurs, therefore, the display controller 11 causes interest information corresponding to arrythmia to be displayed in at least one of the display 5 and the display 6, while highlighting the information (for example, the display is caused to blink, the color is changed, or the font is made larger than usual), in accordance with the tenth control rule.

At this time, in the case where an event in which the ultrasonic image S is partly is changed because of bleeding or the like occurs, the display controller 11 may cause interest information of bleeding/non-bleeding detected from the ultrasonic image S to be displayed in at least one of the displays 5 and 6 while highlighting the information, in accordance with the tenth control rule.

As described above, in the case where an event indicating an abnormality of the subject such as that to be notified by the notifying section 23 occurs, the display controller 11 causes interest information corresponding to the event to be displayed while being highlighted. Therefore, the user can quickly know the contents of the notification, and adequately cope with the abnormal event. An alarm sound notifying the event may be output in the same or similar manner as in a usual patient monitor.

According to the embodiment, in the case where a predetermined event occurs, the display controller 11 causes interest information corresponding to the predetermined event to be displayed in at least one of the displays 5 and 6, and therefore the user can quickly cope with the predetermined event.

### Embodiment 3

In Embodiments 1 and 2 described above, preferably, the display controller 11 causes information including the vital signs V to, in the display 5 and the display 6, be displayed in the display 5 with a higher predetermined priority.

For example, a priority relationship in which the priority of the display 5 is higher than that of the display 6 may be previously set in the display controller 11. The priorities can be set based on the characteristic information of the displays 5 and 6 such as the sizes of the screens, the installation places, and the usage purposes, or arbitrarily set by the user.

As described above, information including the vital signs V is displayed in the display 5 with the higher priority, and, in the case where the user wishes to check the vital signs V, the user is required only to view only the display 5. Hence, it is possible to know more quickly the vital signs V.

According to the embodiment, the display controller 11 causes information including the vital signs V to be displayed in the display 5 with a higher predetermined priority, and therefore it is possible to know more quickly the vital signs V.

### Embodiment 4

In Embodiments 1 to 3 described above, the display controller 11 may cause the display contents of at least one of the vital signs V and the ultrasonic image S to be displayed separately and respectively in the display 5 and the display 6.

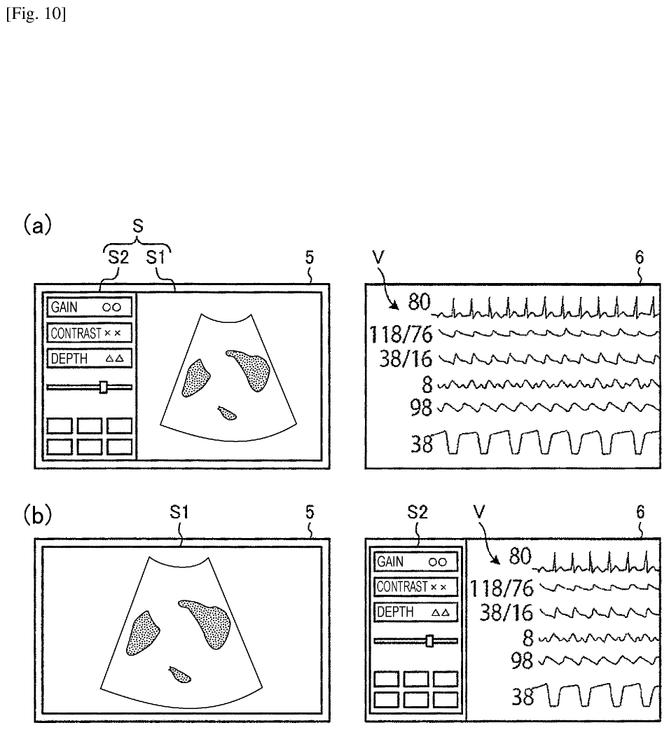

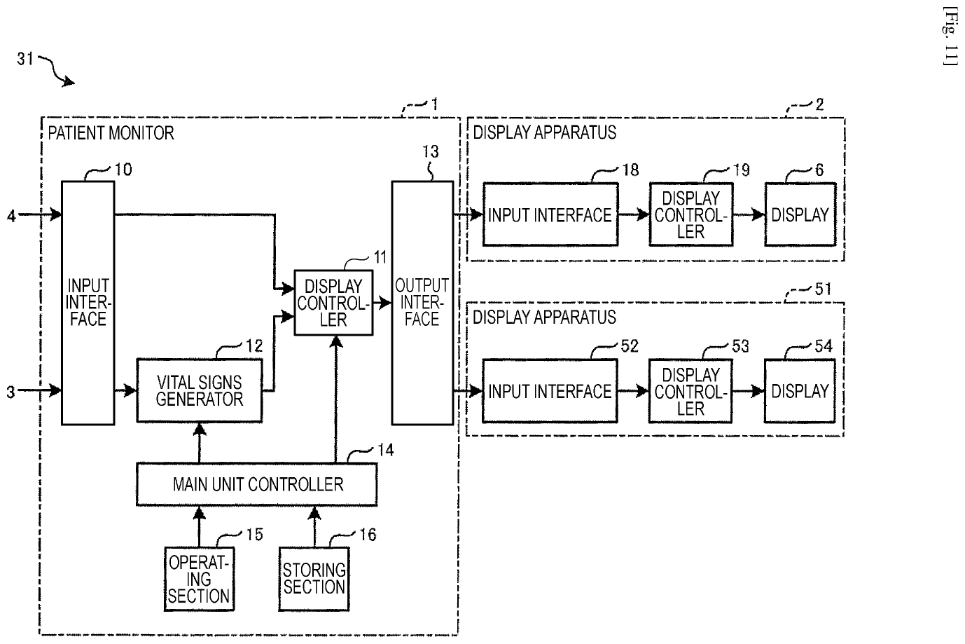

In the case where, as illustrated in FIG. 10(a), the display contents of the ultrasonic image S are configured by a main portion S1 configured by an image itself, and an auxiliary portion S2 for operating the display of the image portion, for example, the auxiliary portion S2 acts as an obstacle, and it becomes difficult to largely display the main portion S1 in the whole display 5.

Therefore, it is preferable that an eleventh control rule for, as exemplarily illustrated in FIG. 10(b), displaying the main portion S1 of the ultrasonic image S in the display 5, and the auxiliary portion S2 in the display 6 is previously set in the display controller 11. This allows the user to cause the main portion S1 to be displayed in the whole display 5, and check in detail the ultrasonic image S. In the case where the display of the image of the main portion S1 is to be operated, the operation can be performed through the auxiliary portion S2 that is displayed in the display 6. Since the display controller 11 causes the display contents of the ultrasonic image S to be displayed separately and respectively in the display 5 and the display 6, therefore, it is possible to display more largely the image itself of the ultrasonic image S.

The display controller 11 can select the displays 5 and 6 in which the main portion S1 and the auxiliary portion S2 are to be displayed, based on information of the displays 5 and 6, for example, the characteristic information of the displays 5 and 6 such as the sizes of the screens, the installation places, and the usage purposes.

According to the embodiment, the display controller 11 causes the display contents of at least one of the vital signs V and the ultrasonic image S to be displayed separately and respectively in the display 5 and the display 6, and therefore it is possible to largely display desired display contents.

Although, in Embodiments 1 to 4 described above, the display controller 11 is connected so as to control the two the displays 5 and 6, the display controller is requested to control a plurality of displays, and the number of displays is not limited to two.

Although, in Embodiments 1 to 4 described above, the display 5 is incorporated in the patient monitor 1, the display is requested to be configured so as to be connectable to the display controller 11, and the configuration is not limited to this. As illustrated in FIG. 11, for example, the display 5 may be omitted in Embodiment 1, and a display apparatus 51 may be newly disposed. The display apparatus 51 is configured in the same or similar manner as the display apparatus 2, and an input interface 52, a display controller 53, and a display 54 are sequentially connected to the output interface 13 of the patient monitor 1.

According to the configuration, in the same or similar manner as Embodiment 1, the display controller 11 is controllably connected to the displays 6 and 54, and can control the displays 6 and 54 so as to display information including the vital signs V in at least one of the displays 6 and 54. In this way, the display 54 is placed separately from the patient monitor 1.

Although, in Embodiments 1 to 4 described above, the ultrasonic image generator 8 is incorporated in the ultrasonic measurement apparatus 4, the ultrasonic image generator is requested to produce the ultrasonic image S, and the configuration is not limited to this. For example, the ultrasonic image generator 8 may be incorporated in the patient monitor 1, and connected between the input interface 10 and the display controller 11. Namely, the ultrasonic measurement apparatus 4 acquires the receiving signals of the ultrasonic echo that function as the base of an ultrasonic image, and outputs the receiving signals to the patient monitor 1, and the ultrasonic image is produced by the ultrasonic image generator 8 incorporated in the patient monitor 1.

Although, in Embodiments 1 to 4 described above, the vital signs generator 12 is incorporated in the patient monitor 1, the vital signs generator is requested to produce the vital signs V, and the configuration is not limited to this. For example, the vital signs generator 12 may be placed separately from the patient monitor 1, and connected between the sensor 3 and the input interface 10.

Although, in Embodiments 1 to 4 described above, the patient monitor 1 is connected to the display apparatus 2, the sensor 3, and the ultrasonic measurement apparatus 4 through the cable lines, they may be wirelessly connected to each other.

In Embodiments 1 to 4 described above, the patient monitor 1 may include the ultrasonic measurement apparatus 4 and the sensor 3.

In Embodiments 1 to 4 described above, the patient monitor 1 may be configured so as to include a plurality of display apparatuses.

The present application is based on Japanese Patent Application No. 2019-008780 filed on Jan. 22, 2019, the entire contents of which are hereby incorporated by reference.

1. A patient monitor for acquiring vital signs that are based on vital signals of a subject, and an ultrasonic image that is based on an ultrasonic wave which is transmitted to and received from the subject, wherein
the patient monitor includes a display controller that is configured to be connectable to a plurality of displays, and that is configured to control the plurality of displays to display information including the vital signs in at least one of the displays.

2. The patient monitor according to claim 1, wherein the display controller is configured to cause information including the ultrasonic image to be displayed in another display other than a display displaying the information including the vital signs.

3. The patient monitor according to claim 1, wherein, in a case where a predetermined event occurs, the display controller is configured to cause interest information corresponding to the predetermined event to be displayed in at least one display of the plurality of displays.

4. The patient monitor according to claim 3, wherein the display controller is configured to change information displayed in the plurality of displays so as to correspond to the predetermined event.

5. The patient monitor according to claim 3, wherein the display controller is configured to cause the interest information corresponding to the predetermined event to be highlighted in at least one display of the plurality of displays.

6. The patient monitor according to claim 3, wherein the predetermined event includes at least one of variation of the vital signals partial variation of the ultrasonic image, operation of an operating section, connection and disconnection of an ultrasonic measurement apparatus, connection and disconnection of the displays, elapse of a predetermined time period, and reception of data from an external apparatus.

7. The patient monitor according to claim 1, wherein the patient monitor incorporates at least one of the displays.

8. A physiological information measurement system including:
an ultrasonic measurement apparatus configured to acquire receiving signals of an ultrasonic echo which is a base of an ultrasonic image, or an ultrasonic image; and
the patient monitor according to claim 1.
